(19) United States
(12) Patent Application Publication
(10) Pub. No.: US 2022/0087535 A1
(43) Pub. Date: Mar. 24, 2022

(54) METHOD FOR DETERMINING A STATE OF OVER-HEATING OR A RISK OF OVER-HEATING OF A SUBJECT

(71) Applicant: WELL BEING DIGITAL LIMITED, New Territories (HK)

(72) Inventor: Ming Yip Wallace WONG, New Territories (HK)

(21) Appl. No.: 17/425,669

(22) PCT Filed: Jan. 6, 2020
(86) PCT No.: PCT/CN2020/070515

§ 371 Date: Jul. 23, 2021

(30) Foreign Application Priority Data
Jan. 23, 2019 (HK) 19101200.3

(51) Int. Cl. A61B 5/01; A61B 5/00

(52) U.S. Cl. CPC A61B 5/01; A61B 5/6817; A61B 5/7275; A61B 5/0008; A61B 2562/043; A61B 5/746; A61B 2560/0223; A61B 2562/0276; A61B 5/7405

(57) ABSTRACT

A device to be inserted into the ear canal having at least two thermistors for observing a temperature gradient in the ear canal of a user. Changes in the temperature gradient are monitored for determining the risk of the user in facing an imminent heat stroke. The device includes a plug suitable for restricting air flow through the opening of the ear canal, a first temperature monitor arranged to measure the temperature of air restricted in the ear canal at a first location in the ear canal, and a second temperature monitor arranged to measure the temperature of air restricted in the ear canal at a second location in the ear canal. The second location being deeper in the ear canal than the first location.

### FIELD OF INVENTION

This invention relates to the field of continuous monitoring of bodily temperature. In particular, the invention relates to apparatuses and methods for monitoring danger caused by or accompanied by changes in bodily temperature, such as heatstroke.

### BACKGROUND OF THE INVENTION

Conventionally, body temperature is measured using a mercury thermometer. The mercury thermometer has a glass bulb filled with mercury which overflows from the bulb into a capillary tube. The mercury expands and contracts in the capillary tube according to heat transmission into or away from the mercury across the bulb wall. The bulb is placed against the body of a subject whose temperature is to be measured, and is typically inserted into a crevice of the body, such as beneath the tongue, in an arm pit, or into the rectum. The choice of location depends on the age of the subject. Time is required for heat to transfer from the body into the mercury and to reach equilibrium so that expansion of the mercury stabilises. Hence, it is often time consuming and uncomfortable for the subject who is inserted with a mercury thermometer to wait while his temperature is being read.

The mercury thermometer suffers from an important disadvantage in that it cannot be used to monitor the subject's temperature over a period of time continuously. The mercury thermometer is only useful for providing a single reading of temperature, of a discrete moment.

The tympanic infrared thermometer has been proposed, which measures body temperature more comfortably by detecting infrared emissions from the ear drum. The ear drum is also known as the tympanic membrane. The tympanic infrared thermometer is commonly seen as a handheld device in clinics, and has a spout containing an optical detector. The spout is shaped to be inserted into the earhole. The optical detector detects infrared emissions from the tympanic membrane, and the tympanic infrared thermometer very quickly deduces body temperature from the emissions based on calibration. An advantage of the tympanic infrared thermometer is that body temperature is read very quickly, virtually in a second. This relieves the subject of the need to wait while his temperature is being read, unlike using the mercury thermometer. However, it is difficult to provide a line of sight from the optical detector at the opening of the ear hole to the tympanic membrane, especially if the handheld device is not handled skillfully. Furthermore, such a handheld device is not designed to be worn by a subject over a period of time, and is therefore not useable for continuous body temperature monitoring. Like the mercury thermometer, the handheld tympanic infrared thermometer is only useable to obtain a single temperature reading.

It has been a desire for subjects who are firemen to have their temperature monitored during their training or work, in order to monitor their risk of serious heat injury. In a hot environment with intense work stress, a fireman is unlikely to notice that he is running a fever and that he is in danger of heat injury. It is also difficult for his supervisor, who is usually nearby conducting the fire rescue carried out by the fireman but distanced from the fire itself, to monitor the fireman's condition by relying on observation skills of other firemen in the team. If a fireman collapses from heat injury, his team mates will have to turn to focus on rescuing him instead of fighting fire.

It has been proposed to configure the tympanic infrared thermometer into an ear-wearable design. A fireman can then wear it in one of his ears during fire rescue, so that his temperature may be monitored continuously throughout the rescue. However, the ear-wearable design suffers the same inherent difficulty of providing a line of sight between the optical detector and the tympanic membrane. Moreover, when worn over a period of time, it is likely that movements of the fireman can eventually break the line of sight.

Any device that monitors body temperature accurately and precisely is said to be a ‘sensitive’ thermometer, and has to be calibrated in order to be accurate and precise. However, calibration is subject to drifting. This imposes a need for regular re-calibration to maintain accuracy. If a sensitive device is used in a busy situation that subjects the device to the force of a lot of movements, sudden and significant calibration drifts may occur. If a sensitive thermometer is relied upon to raise an alarm when the subject's temperature is too high, a calibration drift could cause false alarm or cause a valid alarm not to be raised. Hence, overly sensitive thermometers are not suitable for use in continuous monitoring of firemen's temperature during fire rescues.

It has also been proposed to provide a telemetric pill which can be swallowed by a subject to provide accurate body temperature monitoring over a period of time. A swallowed telemetric pill travels through the gut without being subject to high impact forces, and measures the subject's body temperature accurately as long as it remains inside the subject. The telemetric pill wirelessly transmits temperature readings to a device external to the subject's body in order that the readings may be displayed. However, the telemetric pill is very expensive and not suitable for re-use due to hygiene and decency concerns.

Accordingly, it is desirable to provide an improved device and method which could provide a period of continuous monitoring of body temperature, and possibly identify a risk of imminent heatstroke and also mitigate the aforementioned problems.

### STATEMENT OF INVENTION

In a first aspect, the invention proposes a method for determining a state of over-heating or a risk of over-heating of a subject, or user, comprising the steps of obtaining the temperature gradient of an ear canal of the subject; detecting a change in the temperature gradient; and determining a state of over-heating, or risk of over-heating, if the change in temperature gradient is beyond a pre-determined threshold level.

The temperature gradient is typically along the ear canal.

By observing for a change in temperature gradient to determine a state of over-heating, or risk of over-heating, this method provides a possibility that exact body temperature measurement may no longer be required to make that determination. The method may therefore be applied in a device for relatively rugged use, such as in monitoring the heat condition of a fireman or worker in a hot working environment, making the device robust.

Preferably, the method further comprises the steps of providing a first temperature monitor at a first location in the ear canal of the subject and a second temperature monitor at a second location in the ear canal, the second location being deeper in the ear canal than the first location; wherein, the first temperature monitor and the second temperature monitor each monitoring the temperatures of the air in the ear canal at respective locations to provide an observation of a temperature gradient.

Preferably, the step of determining a state of over-heating or risk of over-heating if the change in temperature gradient is beyond a pre-determined threshold level comprises: obtaining an initial temperature gradient of the ear canal, and referencing the change in temperature gradient against the initial temperature gradient.

Referencing change in temperature gradient against the initial temperature gradient provides that the initial condition of the subject is taken as the reference to evaluate whether any change in core body temperature is a cause for concern. In other words, whatever the initial body temperature of any given period of temperature monitoring may be, it is assumed to be normal body temperature. As the point of reference is this initial condition, there is no need for exact temperature measurement; the extent of departure from this initial condition is possibly sufficient for founding an assumption on the seriousness of an increase in core body temperature. By removing the need of knowing the body temperature accurately before risk of heat injury may be determined, the invention has made progress contrary to conventional wisdom and industrial tendencies.

Typically, the method further comprises the steps of: requiring an observation of a steepening of the temperature gradient; and requiring an observation of an increase in temperature of the air at the second location in the ear canal. This feature requires that the steepening of the temperature gradient is accompanied by an increase in core body temperature, before the change in temperature gradient may be determined to be indicative of overheating. Accordingly, this feature prevents false alarms when the gradient steepens but not due to an increase in core body temperature, such as because the body temperature has dropped instead of risen. In some circumstances, however, this feature may not even be required, such as if it is certain that any change in the subject's temperature will almost certainly be due to overheating. For example, if the method is applied in a device which is intended to be used on a fireman in a fire rescue. Also, this feature may not be required in some embodiments that monitor hypothermia, instead of overheating.

Alternatively, the method further comprises the steps of: requiring an observation of a steepening of the temperature gradient; requiring an observation of an increase in temperature of the air at the second location in the ear canal; and requiring an observation of an increase in temperature of the air in the first location in the ear canal. Again, this feature possibly prevents false alarms when the gradient steepens. More specifically, this feature identifies steepening of the gradient due to cold ambient temperature causing better heat dissipation from the pinna, which causes a drop in the temperature at the opening of the ear canal. Hence, this feature provides that only when the steepening of the temperature gradient is accompanied by both an increase in the temperatures of the air in both the first location and second location may over-heating or risk of over-heating in a subject be determined.

Preferably, the step of obtaining the temperature gradient of an ear canal of the subject further comprises: sending the temperature obtained by the first temperature monitor and the temperature obtained by the second temperature monitor to a remote device to deduce the temperature gradient.

More preferably, the step of sending the temperature obtained by the first temperature monitor and the temperature obtained by the second temperature monitor to the remote device is done wirelessly.

Typically, the temperatures of the air in the ear canal at the respective locations observed by the first temperature monitor and the second temperature monitor are fitted to a linear model. Alternatively, however, the temperatures of the air in the ear canal as observed by the temperature monitors may be fitted to a non-linear model, such as a curve. In this case, preferably, there is a third temperature monitor to provide a third observation in order to construct the curve with three observations. Possibly, a “change in temperature gradient” may mean change in the curvature of the model when the temperature as read by each of the three thermistors has changed.

In a further aspect, the invention proposes a device for observing temperature in an ear canal of a subject, comprising a plug suitable for restricting air flow through the opening of the ear canal; at least two a first temperature monitors: a first temperature monitor arranged to measure the temperature of air restricted in the ear canal at a first location in the ear canal; a second temperature monitor arranged to measure the temperature of air restricted in the ear canal at a second location in the ear canal; and the second location being deeper in the ear canal than the first location.

Preferably, the device further comprises an extension extending from the plug; the first temperature monitor and the second temperature monitor being located on the extension.

Optionally, the at least two thermistors are comprised in three thermistors, in which case the at least three thermistors are arranged on the extension.

Optionally, the extension cantilevers from the plug to be eccentrically positioned in the ear canal when the device is worn.

Preferably, the extension is narrower than the average ear-canal. Specifically, it is preferable that the extension has a diameter smaller than the diameter of the average ear-canal. For example, the extension has a diameter less than 0.5 centimetres. More preferably, the extension has a diameter less than 0.3 centimetres. This possibly provides that the extension does not contact the ear canal wall, which makes it more comfortable for the subject to bear having the extension inserted into his ear for a long period of time.

Preferably, the extension has a length of less than 1 cm. This reduces the likelihood that the extension abuts against a bend in the ear canal, according to the average ear canal, providing greater comfort for long term wearing, which improves the possibility of continuous monitoring of subject.

Preferably, the device further comprises a speaker. This allows communication to be made to the subject wearing an embodiment of the invention, and for the embodiment to function also as an earphone, or a hearing aid.

Optionally, the temperature monitors are comprised in at least three temperature monitors; a third temperature monitor arranged to measure the temperature of air restricted in the ear canal at a third location in the ear canal; and the third location being between the first location and the second location. This feature allows the temperature gradient to be non-linear, in which case change in gradient may include change in curvature of the temperature gradient.

### BRIEF DESCRIPTION OF THE FIGURES

It will be convenient to further describe the present invention with respect to the accompanying drawings that illustrate possible arrangements of the invention, in which like integers refer to like parts. Other arrangements of the invention are possible, and consequently the particularity of the accompanying drawings is not to be understood as superseding the generality of the preceding description of the invention.

FIG. 1 shows a comparative prior art example to an embodiment of the invention;

FIG. 2 is a schematic diagram of an embodiment of the invention;

FIG. 3 is a schematic diagram of the internal parts of the embodiment of FIG. 2;

FIG. 4 shows how the embodiment of FIG. 2 is put in use;

FIG. 5 shows how the embodiment of FIG. 2 is positioned in the ear;

FIG. 6 is a magnified drawing showing how the embodiment of FIG. 2 is positioned in the ear;

FIG. 7 shows a linear relationship model to which temperatures read by the embodiment of FIG. 2 may be fitted;

FIG. 8 shows how the gradient of the model of FIG. 7 may change;

FIG. 9 also shows how the gradient of the model of FIG. 7 may change;

FIG. 10 also shows how the gradient of the model of FIG. 7 may change;

FIG. 11 also shows how the gradient of the model of FIG. 7 may change;

FIG. 12A is a flowchart showing the deliberations during the operation of the embodiment of FIG. 2;

FIG. 12B shows a variation of the embodiment of FIG. 2 used to obtain the body temperature of the user;

FIG. 13 is a variation of FIG. 12B;

FIG. 14 is a magnified drawing showing how a variation of the embodiment of FIG. 2 is positioned in the ear;

FIG. 15 is a drawing of the pinna, showing the concha;

FIG. 16 shows how a variation of the embodiment of FIG. 2 is used on the pinna shown in FIG. 15;

FIG. 17 shows a variation of the embodiment of FIG. 2 which is part of a hearing aid; and

FIG. 18 shows a further variation of the embodiment of FIG. 2 which monitors hypothermia.

### DETAILED DESCRIPTION OF EMBODIMENTS

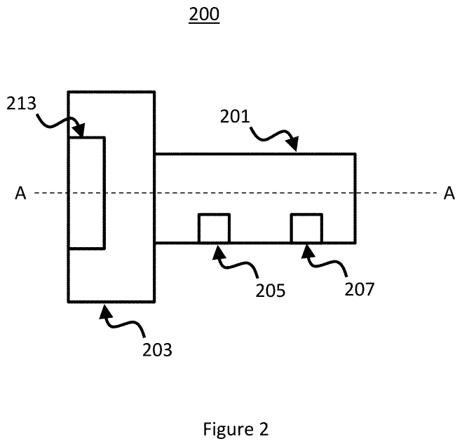

FIG. 2 shows an embodiment 200 of the present invention, which is a device 200 that may be inserted into the opening of an ear canal. The embodiment 200 comprises a plug 203, and an extension 201 or elongate member 201 extending from the plug 203. The user holds the embodiment 200 by the plug 203 when the user inserts the extension 201 into one of the user's ear canals.

The plug 203 has dimensions suitable for fitting into the opening of the ear canal. Preferably, the plug 203 is made of a soft, deformable material such as rubber, silicon or some other kinds of polymers which can deform in order to squeeze into the opening of the ear canal and stay there securely. A good fit restricts or reduces flow of air into and out via the opening of the ear canal. Consequently, this reduces air exchange with the surroundings. The side of the plug 203 facing away from the extension 201 is installed with an LED (light emitting diode), which flashes if an alarm is raised when the user wearing the embodiment 200 is in risk of core temperature over-heating or a heatstroke.

Along axis AA of the extension 201 shown in FIG. 2 are placed two thermistors 205, 207. The thermistors measure temperatures of the air in the ear canal, in different locations, typically in Celsius or Fahrenheit. In other embodiments, thermocouples, any types of miniature thermometers or temperature monitors may be used instead of thermistors. The thermistors 205, 207 are spaced apart from each other along the extension 201. The positions of the thermistors 205, 207 on the extension 201 are pre-determined; the distance of each of the thermistors 205, 207 from the plug 203 is known, as well as the distance Δx between the thermistors 205, 207. Preferably, the thermistors 205, 207 are placed on the same side of the axis of the extension 201, such that the thermistors 205, 207 face roughly the same direction.

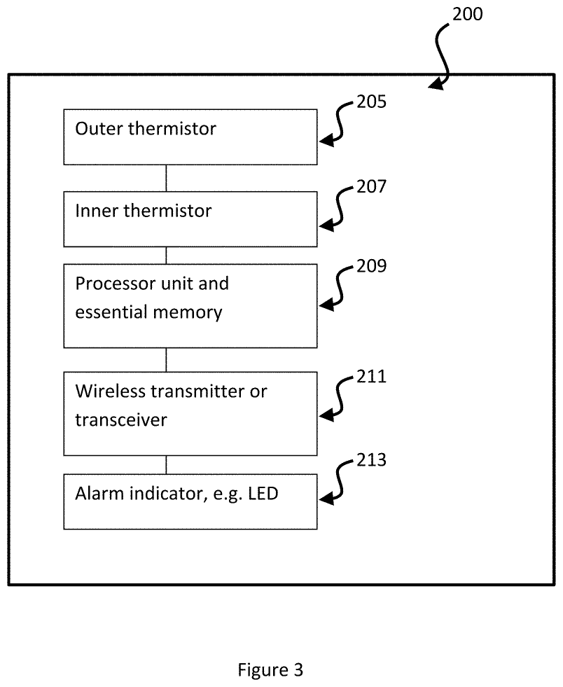

FIG. 3 schematically illustrates some parts of the embodiment 200 which include, besides the inner thermistor 207 (inner meaning further into the ear canal than the outer thermistor 205 when the embodiment 200 is worn) and outer thermistor 205 (outer meaning nearer the opening of the ear canal than the inner thermistor 207 when the embodiment 200 is worn), a processor and any required memory for the processor to operate 209, a wireless transmitter or transceiver 211 and an alarm 213 to indicate overheating of the user wearing the embodiment 200, which is the LED in this embodiment 200. Optionally, the alarm also includes sonic functions to sound an alarm to the user or to people around the user. Furthermore, the alarm optionally includes a speaker (not illustrated) which plays a pre-recorded message into the ear of the user to warn him of his risk of over-heating, operating in a similar way as an earphone.

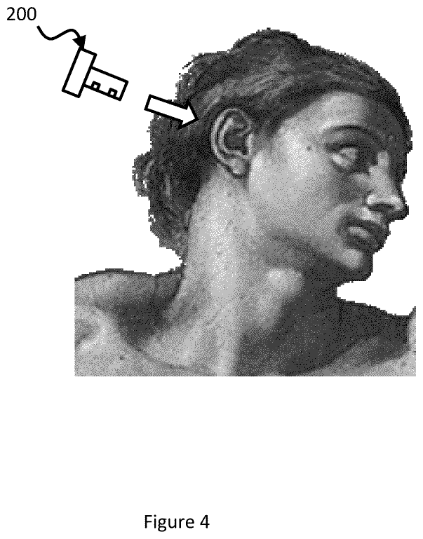

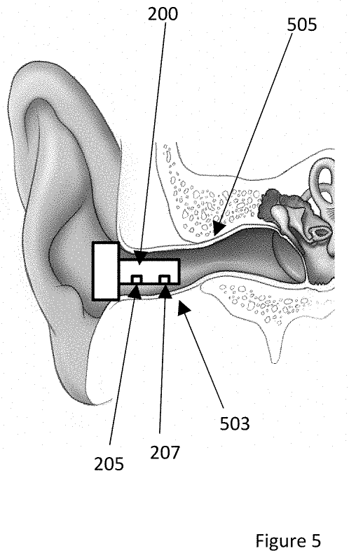

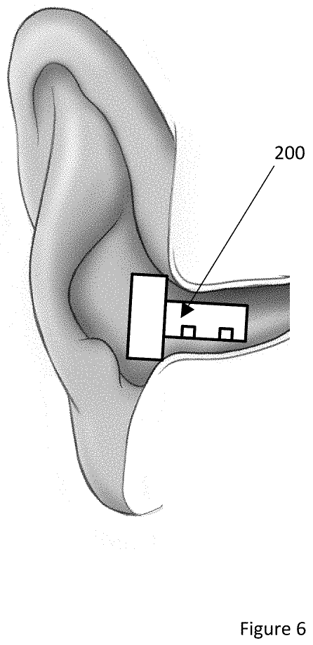

FIG. 4 shows how the embodiment 200 is inserted into the ear canal. Typically, the plug 203 is held by the user's fingers (fingers not illustrated) and the extension 201 is pointed towards the deep end of the ear canal. The plug 203 is then inserted to stop the opening of the ear canal. FIG. 5 is a cross-sectional diagram showing how the embodiment 200 is placed inside the ear canal. FIG. 6 is a magnified part of FIG. 5.

The deformable material which the plug 203 is made of has enough firmness such that the extension 201 is able to cantilever from the inserted plug 203 without resting on or contacting any part of the ear canal wall. When properly inserted, the extension 201 is centrically located in the ear canal and along the axis of the ear canal. On the average, the adult human ear canal extends from the pinna to the eardrum over distance of about 2.5 cm (1″) in length, and has a diameter of 0.7 cm (0.3″). Therefore, the diameter of the extension 201, when regarded axially, is preferably less than 0.7 cm in order to fit into most ear canals. More preferably, however, the diameter is equal to or less than 0.5 cm. This provides that the extension 201 is narrower than most ear canals, increasing the possibility that the sides of the extension 201 are not in contact with ear canal wall. As a result, the user feels only the plug 203 in the opening of the ear canal and possibly not the extension 201. If so, this provides that the embodiment 200 is comfortable enough for the user to wear the embodiment 200 over a long period of time. More importantly, this provides that thermistors 205, 207 measure temperatures of the air in the ear canal and not temperature of the ear canal wall or tissue.

Yet more preferably, the extension 201 has an even smaller diameter at about 0.3 cm, which may provide just enough structural support for the location of the thermistors 205, 207 on the extension 201, and the bluntness of a 0.3 cm extension 201 prevents the tip of the extension 201 from piercing the skin of the ear canal.

The typical ear canal is not a straight passageway. As one can see in FIG. 5, the typical ear canal has two bends, labelled 503 and 505. The first of the bends 503 is rather near the opening of the ear canal. The distance of the first bend 503 from the opening of the ear canal is usually slightly more than 1 cm in most people. Hence, the extension 201 is preferably short enough, at 1 cm or less, to avoid contact with this bend 503.

As the plug 203 generally prevents airflow exchange between ambient air and the air in the ear canal, in a state of equilibrium the temperature of the restricted air in the plugged ear canal is largely due to heat emanating from the core of the body.

Body heat is generated in the body and carried to the skin by flow of blood to be dissipated in the form of bodily radiation and by sweat. Large blood vessels are found deep in the body, carrying most of the warm blood in the body. Some blood in the head near the ear flows towards the pinna, through smaller blood vessels. The skin around the ear and structure of the pinna provides a large surface in which many small capillary blood vessels dissipate heat away from the body quickly. Hence, the temperature of the air in the ear canal near the opening is cooler than the temperature of the air deeper in the ear canal even if the ear canal is plugged. Furthermore, even when temperature at the opening of the ear canal is momentarily higher than the deeper parts of the ear canal, blood in the capillary blood vessels tends to absorb the heat and carry the heat to be dissipated at the skin, thereby cooling the temperature at the ear canal opening. In equilibrium, a relatively stable temperature gradient may be observed.

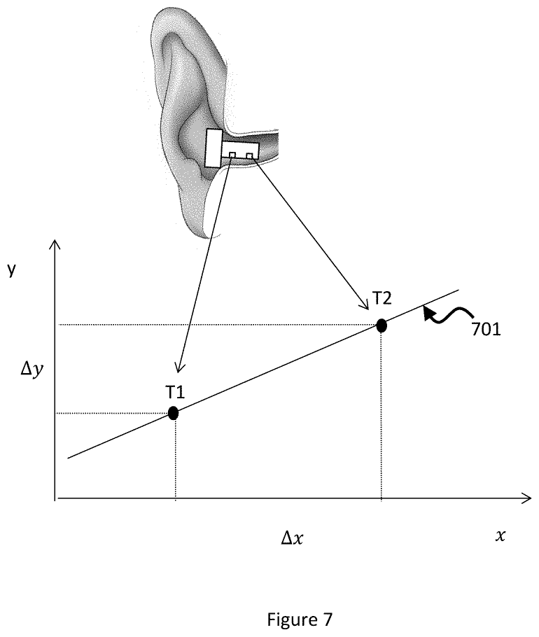

FIG. 7 is a chart showing temperature on the vertical y-axis, and distance in the ear canal on the horizontal x-axis. As illustrated in FIG. 7 and explained above, the temperature of air in the ear canal nearer the opening is lower than the temperature of air deeper in the ear canal. This creates a natural temperature gradient 701 in the ear canal. The thermistors 205, 207 are used to observe this temperature gradient.

As the skilled man knows, temperature gradient is a physical quantity describing the direction and the rate of temperature change. Temperature gradient may be expressed in units of degrees (e.g. Celsius) per unit length, its SI unit being kelvin per meter (K/m), or as expressed as dQ/dt, the rate of heat transfer per second.

However, for simplicity, the temperature gradient in this embodiment 200 is merely expressed as the spread of temperature in the ear canal, Δy, over the physical distance between the inner thermistor 207 and the outer thermistor 205, Δx.

Therefore, assuming the temperature gradient, ∇T, to be linear, it is expressed as follows:

$$∇ T = \frac{Δ y}{Δ x} \qquad ( 1 )$$

As the thermistors 205, 207 are spaced apart along the axis of the extension 201, each of the thermistors 205, 207 measures the temperature of the air in the ear canal in their different, respective locations in the ear canal. Accordingly, temperature which each of the thermistors may detect is different from temperature detected by the other thermistor.

The temperature of the air in the part of the ear canal nearer the opening, measured by the outer thermistor 205, is labelled T1 in the drawings. The temperature of the air in the deeper part of the ear canal, measured by the inner thermistor 207, is labelled T2 in the drawings. In FIG. 7, the temperature gradient of the air in the ear canal is shown increasing from T1 to T2.

The temperatures of the air in the ear canal are unlikely to be the same as the actual temperature of the body. For example, the body may be running a fever at 39° C. but T2 may just be a cooler 32° C. In most temperate and tropical places, the air in the ear canal is normally warmer than the ambient temperature but cooler than the actual body temperature. This is partly because of the relatively lower capacity of air than blood to take up heat, as well as the continuous flow of blood along the ear canal which absorbs away any amount of heat causing the temperature of the air in the ear canal to be greater than the body temperature. That absorbed heat is dissipated by the skin of the pinna to the surroundings outside the ear canal.

Despite the difference between the temperatures of the air in the ear canal and the actual body temperature, the embodiment 200 is able to determine that the user has a life-threatening increase in core body temperature by monitoring for steepening of the temperature gradient. In this way, the embodiment 200 does not require exact measurement of body temperature. This also relieves the need to place the thermistors in precise locations along the ear canal. People with shallower or longer ear canal may use the embodiment to monitor their body heat status, as a temperature gradient may be obtained and observed for changes whether the extension is inserted deeply into the ear canal or not.

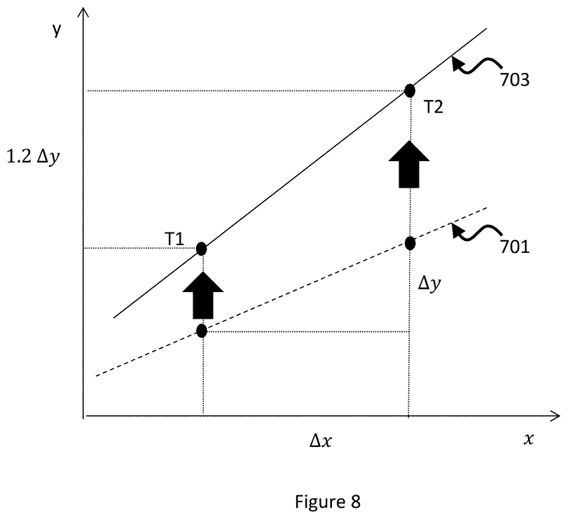

FIG. 8 shows how change in the temperature gradient can be used to determine if the user is overheating. If the core temperature of the user has increased suddenly, such as in a case of an imminent heatstroke, heat inside the body will be generated more quickly than heat may be dissipated by the skin. As a result, the temperature of the air monitored by the inner thermistor 207 in the deeper part of the ear canal, T2, increases. The temperature of the air monitored by the outer thermistor 205 in the part of the ear canal nearer the opening, T1, also increases but to a lesser extent than T2, partly due to the heat dissipative function of the nearby blood, pinna and skin. Eventually, equilibrium is reached and a new temperature gradient 703 having a greater value than the original Δy/Δx, and which is steeper, is observed.

If the extent of the change of the temperature gradient 701 into the new temperature gradient 703 is more than a threshold level, such as 20% more than the original Δy/Δx, the embodiment 200 raises an alarm indicating that the user might be in imminent danger of a heatstroke. In other words, if the new temperature gradient 703 has a value of 1.2×Δy/Δx, as illustrated in the chart of FIG. 8, an alarm that the user is over-heating is raised.

“20%” is an arbitrary example of a threshold given here, and the actual threshold can be determined finally by the manufacturer of a product embodying the invention. Instead of 20%, the actual threshold can be determined by making statistical observations on people, and is beyond need of elaboration for the scope of this description.

Also, 20% refers only to the amount of change of the temperature range Δy, as read between the outer thermistor 205 and the inner thermistor 207. That is, if the original T2 is 30° C. and T1 is 28° C., the 20% increase means a 20% increase on the range of 28° C. to 30° C., or 0.2×2° C., which is just about 0.4° C. That is, if Δy increased by about 0.4° C., the alarm is raised. Therefore, a rise of 20% in the temperature gradient in the ear canal air does not necessarily translate to a 20% increase in actual body temperature.

In practice, after the user puts on the embodiment 200, the temperatures of the air in the respective locations in the ear canal, i.e. T1 and T2, are measured with the ear canal plugged. As soon as T1 and T2 have stabilised, an initial temperature gradient 703 is observed. It does not matter whether the user's normal body temperature is naturally higher or lower than the theoretical normal body temperature. The exact temperature of different normal, healthy individuals actually varies from person to person, and is not always 36.9° C. Subsequently, the embodiment 200 monitors for significant changes in the temperature gradient to determine whether there is a risk of an imminent heatstroke.

As there is no need to obtain the exact temperature of the user's body, the embodiment 200 does not require calibration for interpreting the temperature gradient of the ear canal air into actual body temperature. Not having to operate with exact, accurate temperature reduces the sensitivity requirement of the embodiment 200, making the embodiment 200 robust, not overly-delicate and suitable for deployment in rugged use.

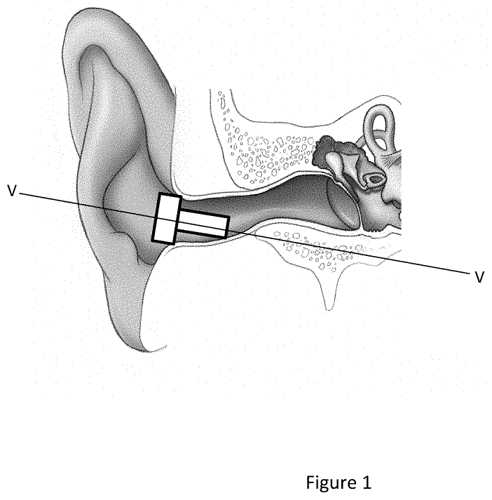

In contrast, if only one thermistor were used to monitor the user's risk of heatstroke, exact body temperature would have to be read and the thermistor would have to be placed deep into the ear canal, to be as near the ear drum as possible. This is because the ear is largely a heat dissipating organ, and the outer ear can be much cooler than the core of the body. This is also the reason why the tympanic infrared thermometer needs to have a line of sight to the tympanic membrane for accurate measurement. An illustration of an ear-wearable tympanic infrared thermometer, as a comparative example, is shown in FIG. 1. The tympanic infrared thermometer is shown off-alignment to the tympanic membrane, pointing instead in the line XX, which prevents reading of accurate body temperature.

Accordingly, the embodiment 200 moves away from the conventional teachings of measuring exact body temperature in order to monitor risk of heatstroke, and also does not require line of sight to the tympanic membrane, which is unlike the tympanic infrared thermometer. Hence, any misalignment of the present embodiment 200 to the central axis of the ear canal is unlikely to reduce the effectiveness of the embodiment 200 to raise an alarm to a risk of heatstroke.

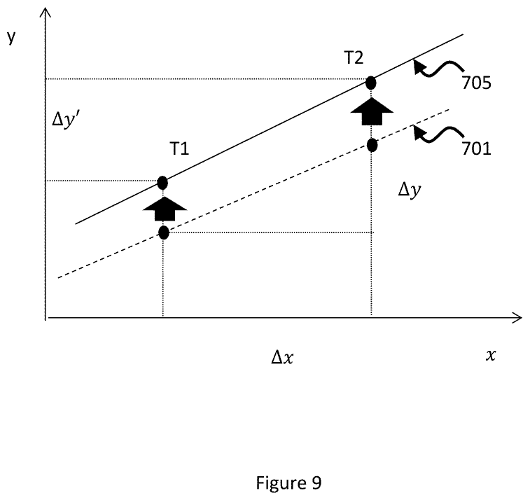

FIG. 9 shows how a user's core body temperature can rise in some circumstances, even though the user is not in danger of heatstroke. Such circumstances must be distinguished from other circumstances which carry a risk of heatstroke. When the user wearing the embodiment 200 engages in strenuous activities, T2 increases due to the rise in core body heat. T1 also increases but only to a smaller extent as heat is dissipated effectively at the pinna by the skin, in the form of radiation and by sweating. In other words, heat is dissipated away fast enough. Accordingly, it is observed that there is little change in the magnitude of Δy′ compared to Δy, and the new temperature gradient 705 in this case has not steepened very much from the original temperature gradient 701. As the slight change in temperature gradient does not reach the extent of a pre-determined threshold, the alarm to warn of a heatstroke is not raised.

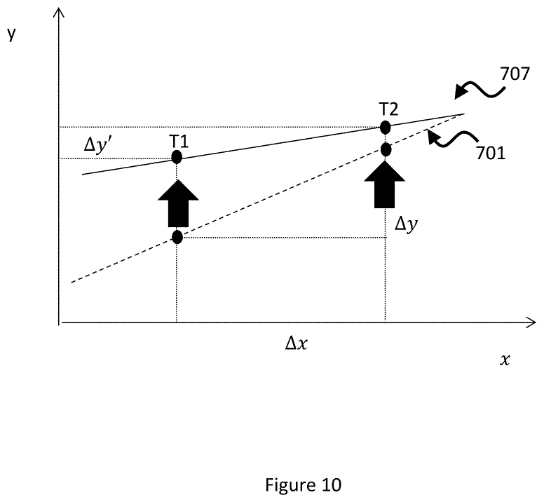

FIG. 10 shows a situation in which the temperature gradient changes to become gentler instead of steeper. This occurs when the user steps into an environment where the ambient temperature is hotter from another environment where the ambient temperature is cooler and, as a result, his body heat is not as effectively dissipated as in the earlier environment. However, the user remains able to tolerate the surrounding heat because his core body temperature has not increased much. As shown in FIG. 10, T1 increases significantly and T2 increases just a little or does not change, and magnitude of Δy′ of the new temperature gradient 707 reduces compared to Δy of the original temperature gradient 701. In this case, because the temperature gradient has become gentler, the alarm to warn of a heatstroke is not raised.

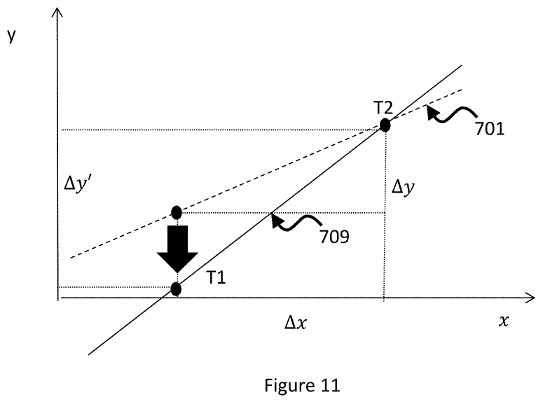

Preferably, to determine that there is an imminent danger of heatstroke further requires both the two thermistors to detect a rise in the temperatures of the air in their respective locations in ear canal. In other words, there is a positive increase of both T1 and T2 besides steepening of the temperature gradient. FIG. 11 illustrates this case. This is to avert false alarm caused by a steepening of the temperature gradient which is due only to reduction in T1. Such a reduction of T1 may be caused by the plug 203 not stopping airflow into and from the ear canal sufficiently, and cold ambient air interacts with the air in the ear canal near the opening, or may be caused simply because the ambient temperature is extremely cold. When T1 decreases but T2 does not change, a new temperature gradient 709 which is steeper will be observed. This is because Δy′ of the new temperature gradient 709 is greater than Δy of the original temperature gradient 701.

Therefore, to distinguish this harmless steepening of the temperature gradient from the kind of steepening which accompanies a heatstroke, the alarm to warn of a heatstroke is not raised if T1 and T2 did not both increase.

Optionally, the temperatures measured by both thermistors 205, 207 are sent wirelessly to a remote computing device or server to deduce the temperature gradient. This is to reduce data processing in the embodiment 200 as much as possible, especially if the embodiment 200 is worn by a user who is fireman in a hot, fire rescue situation. Less tasks for the processor to execute means the embodiment 200 is able to operate more efficiently and with less energy consumption. Alternatively, the temperatures measured by both thermistors 205, 207 are compiled into a temperature gradient by a processing device inside the embodiment 200. Information on the threshold of temperature gradient change is pre-stored in the processor's memory. The processor is thereby able to check at any time if the extent of change in the temperature gradient has reached the pre-determined threshold.

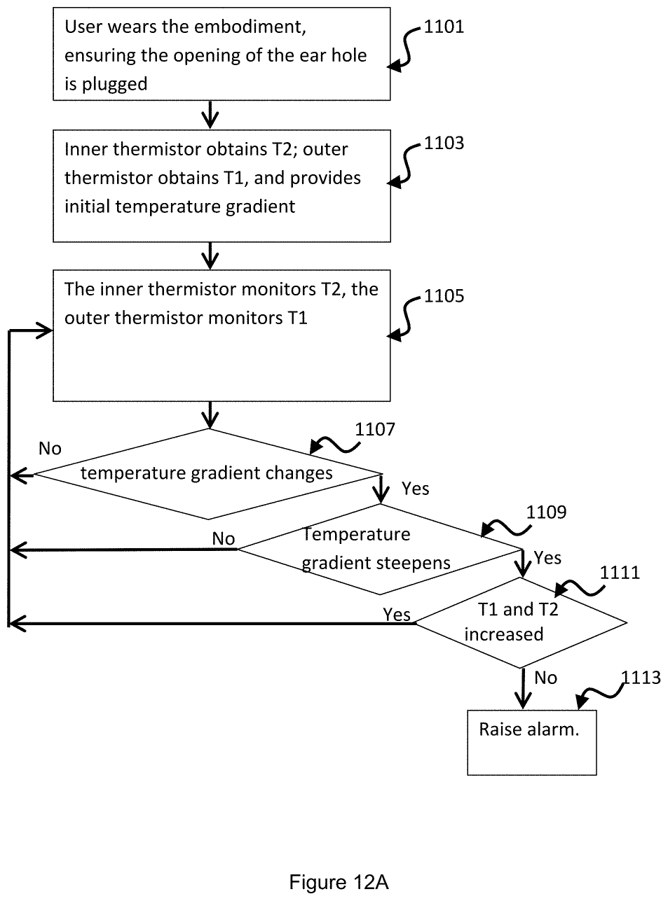

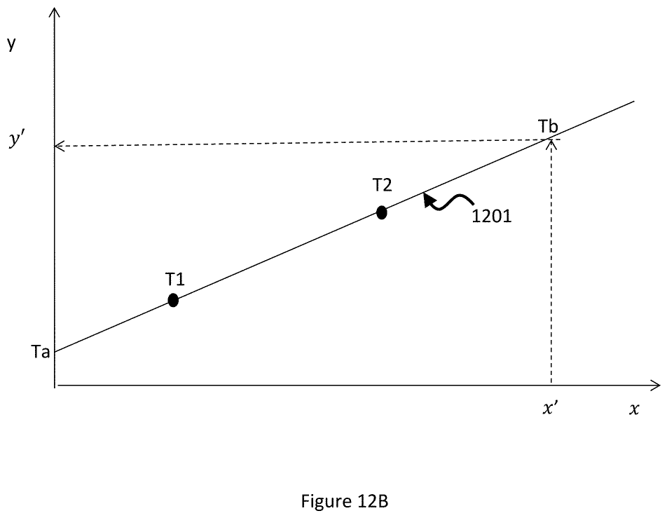

FIG. 12A is a flowchart corresponding to the situations illustrated in FIG. 8 to FIG. 11, showing how the embodiment 200 is used to determine if the user is in imminent danger of heatstroke.

In step 1101, the user inserts the embodiment 200 into his ear. The plug 203 stops air in the earhole from mixing with ambient air. In step 1103, the outer thermistor 205 measures T1 in a part of the ear canal nearer the opening of the ear canal, while the inner thermistor 207 measures T2 in a deeper part of the ear canal. A temperature gradient 701 is observed when the temperature of the air in the ear canal has stabilized. At this point, as the user has just put on the embodiment 200 into his ear, his body temperature at this very instant is assumed to be normal, i.e. typically deemed 36.9° C. This is because, if the user is a fireman about to fight a fire, he is unlikely to be running a fever already. Hence, the initial condition of the user is taken to be the reference against which he will be monitored for deviation therefrom. In other words, whatever temperature gradient is observed in the ear canal when the user first puts on the embodiment 200 will be deemed the reference temperature gradient or original temperature gradient 701, against which gradient change is observed, compared and evaluated. The original temperature gradient 701 is obtained afresh every time the user wears the embodiment 200 anew.

At step 1105, the thermistors 205, 207 monitor the temperatures of the air in the ear canal continuously. If no change in temperature gradient is observed, at step 1107, the thermistors 205, 207 simply continue, at step 1105, to monitor the temperatures of the air in the ear canal. If a change in the temperature gradient in the ear canal is observed, at step 1107, then the next step is to determine, at step 1109, if the temperature gradient has steepened compared to the original temperature gradient 701, or has become gentler.

If it is determined, at step 1109, that the temperature gradient has not steepened sufficiently or has become even gentler in the direction of T1 to T2, the thermistors 205, 207 returns to monitoring the temperatures of the air in the ear canal, at step 1105. There is no need to raise any alarm.

On the other hand, if it is determined, at step 1109, that the temperature gradient has significantly steepened in the direction of T1 to T2, reaching the pre-determined threshold, the next step is to determine if both thermistors 205, 207 observe an increase in temperature. That is, whether T1 and T2 have both increased. This ensures that the false alarm as described in FIG. 11 is not raised. Therefore, if it is determined, at step 1111, that both T1 and T2 have increased, an alarm is raised, at step 1113, to warn that user that he is at risk of a heatstroke.

Optionally, in some embodiments, even if it is determined that only T2 has increased, but T1 has remained constant, an alarm is also raised to warn that the user is having a risk of heatstroke. This is because an increase in T2 is probably due to increase in core body temperature despite not being accompanied by an increase in T1.

Optionally, if the steepening of the temperature gradient is caused by an increase in T2 (increase in core temperature) but also by a decrease in T1 (probably due to cooler ambient temperature), a stricter threshold may be applied, such as by requiring a 25% increase in the gradient instead of the 20% (given as example above). A higher threshold helps to ensure that there is a real risk of heatstroke before an alarm is raised, and that the significant steepening of the temperature gradient is not caused largely by colder ambient temperature.

If it is determined that requiring an observation of a steepening of the temperature gradient is caused only by a decrease in T1, as described in FIG. 11, then the cause of the steepening of the temperature gradient is due to a cooler ambient environment, and no alarm is raised.

Although embodiments have been described which does not require the exact temperature of the user to be known to raise a heat stroke alarm, it is nevertheless possible in some embodiments to determine the exact body temperature of the user. FIG. 12B shows such an embodiment, in which the temperature gradient 1201 can be used to make an extrapolation to determine the actual temperature, y′, of the user. In FIG. 12B, the ambient temperature is labelled Ta. The user's actual temperature is labelled Tb. Ta and Tb are two points which forms a linear relationship. T1 and T2 are the temperatures of specific locations in the ear canal, as observed by the thermistors 205, 207 and are in line with the relationship between Ta and Tb. Mathematically, they may be expressed as follows:

T1=f(Ta,Tb) (2)

T2=f(Ta,Tb) (3)

Therefore, it is possible to deduce Tb from the relation as supposed by the model, where the tympanic membrane is assumed to be in position x′ in the ear canal. Position x′ can be established for each individual user using any measurement methods, or may simply be estimated.

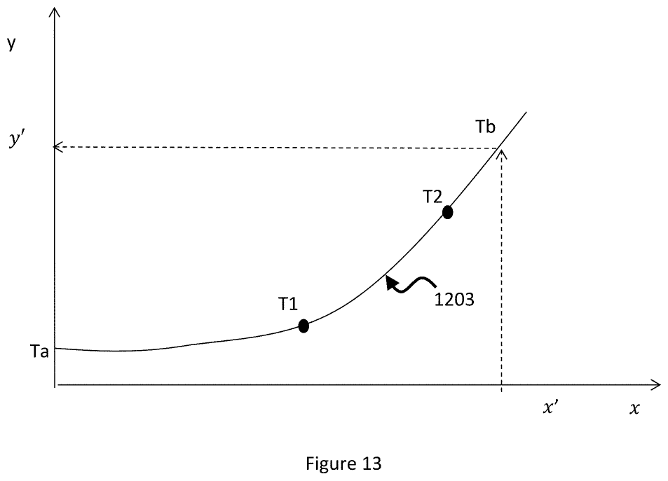

FIG. 13 is a variation of FIG. 12B. While FIG. 12B uses a linear relationship model to predict Tb, FIG. 13 shows the relationship model to be a curve 1203 that extends exponentially. As with the case in FIG. 12B, T1 and T2 are measured by the inner thermistor 207 and the outer thermistor 205, and the model is used to obtain Tb. Any other relationship model can be used. The specific relationship model to use is a choice for the manufacturer of the product embodying the invention to make, which may depend on the brand and make of the thermistors. It is possible to fit the temperature of the air in the ear canal as read by the two thermistors to a pre-selected curved model. Preferably, it is possible to provide more than two thermistors on the extension to read and plot a curved model, i.e. provide at least three points of temperature in the ear canal which spread out in a curve model such as that in FIG. 13a (not illustrated).

Regardless of the choice of model, be it a linear one as shown in FIG. 12B or a curved one as shown in FIG. 13, the relationship can be calibrated to more accurately predict the user temperature. For example, the initial temperature gradient can be calibrated to the user's temperature when he first wore the embodiment 200, by assuming that the temperature is 36.9° C. This would be a one-point calibration. Henceforth, any change in the temperature gradient relies on the calibration to predict the temperature of the user. Specific details of convention calibration methods are well known and do not require elaboration here. In such embodiments in which the actual body temperature of the user is measured, it is an option to raise the alarm to warn of the user being at risk of heatstroke when the body temperature of the user has risen and reached a specific threshold temperature, such as 38° C., instead of relying on an extent of change in temperature gradient to raise the alarm.

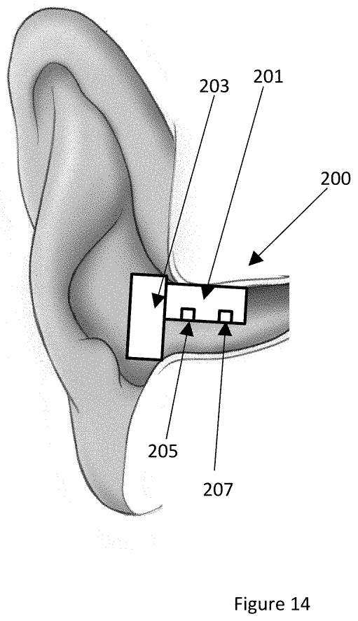

FIG. 14 shows a variation of the embodiment 200, the variation being in the position of the extension 201 on the plug 203. The extension 201 is located on the plug 203 in such a way that when the plug 203 is fitted properly into the opening of the ear canal, the extension 201 is positioned in the ear canal eccentrically. One side of the extension 201 touches the ear canal wall. To ensure that the thermistors 205, 207 do not measure temperature of the ear canal wall and only measure the air in the ear canal, the thermistors 205, 207 are placed on the other side of the extension 201 which is not in contact with the ear canal wall. Advantageously, this allows the user to feel the presence of the extension 201, which lends a sense of security to users who would prefer to know by touch that the extension 201 has been positioned properly.

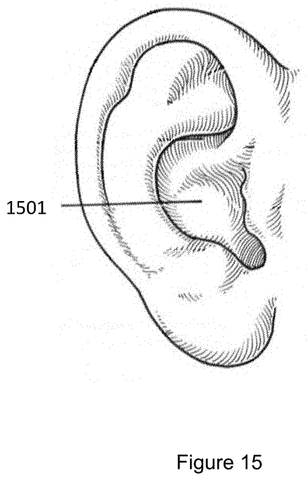

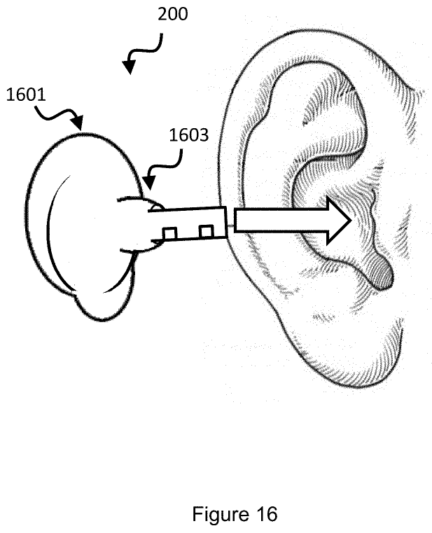

In a preferred variation of the embodiment 200, illustrated in FIG. 16, a part 1601 of the plug 203 has a shape which is moulded to the shape of the concha of an ear of a particular user. FIG. 15 is an illustration of the outer human ear. The concha 1501 is the part of the ear which is a depression just around the opening of the ear canal. The concha has a unique, asymmetrical shape, and varies from user to user. This part 1601 of the plug is usually made of a hard, non-deformable material, such a hard thermoset plastic like Bakelite, glass or fibre glass. The part 1603 of the plug which is to stop the opening of the ear canal is made of a deformable material which can deform to squeeze into the opening of the ear canal. Hence, the plug of this embodiment is made up of a hard, outer part 1601 for the concha, and a soft, inner part 1603 for the opening of the ear canal. Fitting a part of the embodiment 200 to the concha 1501 provides that the position of the plug 203 in the concha and the position of the extension 201 within the ear canal is the same every time the user wears the embodiment 200. This further ensures that the extension 201 is arranged properly in the ear canal and that the thermistors 205, 207 do not touch the ear canal wall.

In another embodiment which is not illustrated, the embodiment is placed within an earphone which is capable of receiving communication information wirelessly such as via Bluetooth™. Such an earphone can be worn by every member in a team of firemen to engage in a dialogue with each other and to coordinate themselves during a fire rescue. If the embodiment determines that any one of the firemen is likely to suffer from a heatstroke, the alarm raised includes an audio message sent to the earphones worn by all the team members.

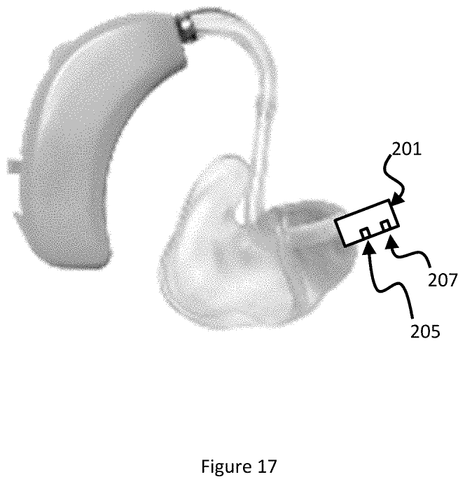

FIG. 17 shows another embodiment 200 which is a hearing aid fitted with an extension 201 having the thermistors 205, 207 as described in the aforementioned embodiments.

As elderly people tend to wear a hearing aid throughout the day, this embodiment 200 allows elderly people to be monitored continuously for increase in body temperature without the elderly people feeling bothered by it. This embodiment is particularly helpful in nursing homes in which private nursing attention is spread thin.

Accordingly, the embodiments include a method for determining a state of over-heating or a risk of over-heating of a subject, i.e. user of the embodiments, comprising the steps of: obtaining the temperature gradient 701 of an ear canal of the subject; detecting a change in the temperature gradient; and determining a state of over-heating or risk of over-heating if the change in temperature gradient is beyond a pre-determined threshold level.

Typically, a subject who is considered as over-heated means his core temperature has risen beyond an acceptable normal level. This does not mean that the subject is already delirious or has suffered a heatstroke, as that would be quite apparent to anyone around him. In most situations, the meaning of the subject over-heating means that the subject's core temperature has raised so high and his ability to dissipate the heat is so bad that he is in danger or risk of suffering injury and immediate treatment should be given to prevent injury, i.e. a stage before serious injury or permanent damage has set in.

Nevertheless, the exact definition of over-heating can be established by each manufacturer of a specific product containing an embodiment of the invention. Over-heating could, for example, be defined to mean that the user has already entered into a state of delirium or heatstroke. Although this would be a less useful threshold as the damage has already set in, a product which detects such a stage may still find some use in setting off a heightened alarm, such as a louder alarm siren from the embodiment than the alarm siren for the subject merely having a risk of imminent heat injury. The heightened alarm indicates greater urgency.

Furthermore, the embodiments include a device 100 for observing temperature in an ear canal of a subject, comprising a plug suitable for restricting air flow through the opening of the ear canal; a first thermistor 205 arranged to measure the temperature of the air in a first position in the ear canal; and a second thermistor 207 arranged to measure the temperature of the air in a second position in the ear canal; the second location being deeper in the ear canal than the first location.

While there has been described in the foregoing description preferred embodiments of the present invention, it will be understood by those skilled in the technology concerned that many variations or modifications in details of design, construction or operation may be made without departing from the scope of the present invention as claimed.

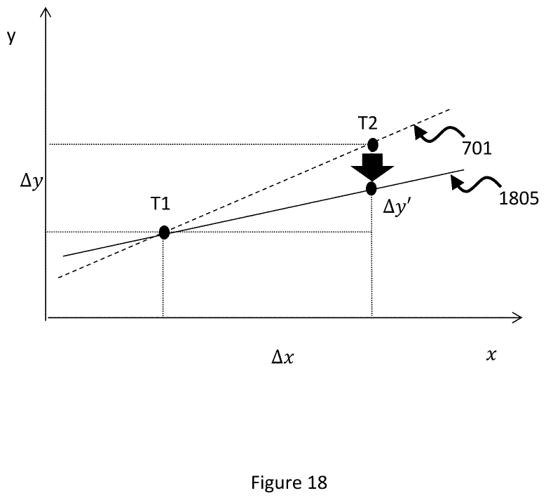

For example, instead of monitoring for risks of over-heating, FIG. 18 shows how the invention may be used in an embodiment which monitors hypothermia, which is a medical emergency that occurs when the body loses heat faster than it can produce heat. In this case, the body turns cold instead of running a fever. In the example given in FIG. 18, the inner thermistor detects that T2 has dropped significantly, which the outer thermistor detects little or no change. The temperature gradient 1805, Δy/Δx, becomes significantly gentler, Δy/Δx. If the temperature gradient 1805 becomes gentler by a certain percentage that exceeds a pre-determined threshold, the alarm is raised to warn of hypothermia. This embodiment is useful for monitoring people who are in cold conditions, such as deep sea divers.

Furthermore, although the user has been described as a person, the embodiments may be applied to animals that require heatstroke monitoring, such as race horses. A horse can be inserted with an embodiment dimensioned and shaped to fit into the horse's ear.

Furthermore, although the thermistors 205, 207 have been described as placed on the same side of the axis of the extension 201, such that the thermistors 205, 207 face roughly the same direction, it is possible that the thermistors 205, 207 face opposite directions on the extension 201 which cantilevers from the plug 203. As long as the thermistors 205, 207 do not contact the ear canal wall, each is able to read the temperature of the air in respective location in the ear canal.

Furthermore, although two thermistors arranged on an extension extending from a plug has been described, variations of the embodiments which include two thermistors, each arranged on a separate extension, each of the extensions extending from the plug and to be inserted into the ear canal, are within the contemplation of this description (not illustrated). In such an embodiment, the first one of the thermistors is arranged on one of the extensions to be in the ear canal but nearer to the opening of the ear hole than the other thermistor, and the other thermistor is arranged on the other one of the extensions to be deeper in the ear canal than the first one of the thermistors.

Furthermore, although the change of temperature gradient in the ear canal has been described as change in the slope of a linear gradient, it is possible that the change may be that of a linear line to a curved line, in which case more than two thermistors are arranged on the extension. There can be as many thermistors as possible on the extension to observe a non-linear, curved temperature gradient. The curve may be exponential, sigmoid or logistic curve, or any other model as the manufacturer of an embodiment deems best suited.

1. A method for determining a state of over-heating or a risk of over-heating of a subject, comprising the steps of:
obtaining the temperature gradient of an ear canal of the subject;
detecting a change in the temperature gradient; and
determining a state of over-heating or risk of over-heating if the change in temperature gradient is beyond a pre-determined threshold level.

2. The method for determining a state of over-heating or a risk of over-heating of a subject as claimed in claim 1, further comprising the steps of:
providing a first temperature monitor at a first location in the ear canal of the subject and a second temperature monitor at a second location in the ear canal, the second location being deeper in the ear canal than the first location; wherein
the first temperature monitor and the second temperature monitor each monitoring the temperatures of the air in the ear canal at respective locations to provide an observation of a temperature gradient.

3. The method for determining a state of over-heating or a risk of over-heating of a subject, as claimed in claim 2, wherein the step of determining a state of over-heating or risk of over-heating if the change in temperature gradient is beyond a pre-determined threshold level comprises:
obtaining an initial temperature gradient of the ear canal;
referencing the change in temperature gradient against the initial temperature gradient.

4. The method for determining a state of over-heating or a risk of over-heating of a subject, as claimed in claim 3, further comprising the steps of:
requiring an observation of a steepening of the temperature gradient; and
requiring an observation of an increase in temperature of the air at the second location in the ear canal.

5. The method for determining a state of over-heating or a risk of over-heating of a subject, as claimed in claim 3, further comprising the steps of:
requiring an observation of a steepening of the temperature gradient;
requiring an observation of an increase in temperature of the air at the second location in the ear canal; and
requiring an observation of an increase in temperature of the air in the first location in the ear canal.

6. The method for determining a state of over-heating or a risk of over-heating of a subject, as claimed in claim 2, wherein the step of obtaining the temperature gradient of an ear canal of the subject further comprises:
sending the temperature obtained by the first temperature monitor and the temperature obtained by the second temperature monitor to a remote device to deduce the temperature gradient.

7. The method for determining a state of over-heating or a risk of over-heating of a subject, as claimed in claim 6, wherein
the step of sending the temperature obtained by the first temperature monitor and the temperature obtained by the second temperature monitor to the remote device is done wirelessly.

8. A device for observing temperature in an ear canal of a subject, comprising
a plug suitable for restricting air flow through the opening of the ear canal;
a first temperature monitor arranged to measure the temperature of air restricted in the ear canal at a first location in the ear canal;
a second temperature monitor arranged to measure the temperature of air restricted in the ear canal at a second location in the ear canal; and
the second location being deeper in the ear canal than the first location.

9. The device for observing temperature in the ear canal of a subject as claimed in claim 8, further comprising
an extension extending from the plug;
the first temperature monitor and the second temperature monitor being located on the extension.

10. The device for observing temperature in the ear canal of a subject as claimed in claim 8, where the extension cantilevers from the plug to be eccentrically positioned in the ear canal when the device is worn.

11. The device for observing temperature in the ear canal of a subject as claimed in claim 8, wherein
the extension has a diameter smaller than the diameter of the ear-canal.

12. The device for observing temperature in the ear canal of a subject as claimed in claim 11, wherein
the extension has a diameter less than 0.5 centimetres.

13. The device for observing temperature in the ear canal of a subject as claimed in claim 11, wherein
the extension has a diameter less than 0.3 centimetres.

14. The device for observing temperature in the ear canal of a subject as claimed in claim 8, wherein
the extension has a length of less than 1 cm.

15. The device for observing temperature in the ear canal of a subject as claimed in claim 8, wherein device further comprises:
a speaker.

16. The device for observing temperature in the ear canal of a subject as claimed in claim 8, wherein
the temperature monitors are comprised in at least three temperature monitors;
a third temperature monitor arranged to measure the temperature of air restricted in the ear canal at a third location in the ear canal; and
the third location being between the first location and the second location.

17.-18. (canceled)
